(19) United States
(12) Patent Application Publication
(10) Pub. No.: US 2022/0246299 A1
(43) Pub. Date: Aug. 4, 2022

(54) ELECTRONIC PATIENT ADVISOR AND HEALTHCARE SYSTEM FOR REMOTE MANAGEMENT OF CHRONIC CONDITIONS

(71) Applicant: OptiChroniX GmbH, Schonenbuch (CH)

(72) Inventor: René M. Gilvert, Schonenbuch (CH)

(21) Appl. No.: 17/586,107

(22) Filed: Jan. 27, 2022

Related U.S. Application Data
(60) Provisional application No. 63/143,588, filed on Jan. 29, 2021.

(51) Int. Cl. G16H 50/20; G16H 40/67; G16H 10/20; G16H 10/60

(52) U.S. Cl. CPC G16H 50/20; G16H 10/60; G16H 10/20; G16H 40/67

(57) ABSTRACT

A system and method for the electronic monitoring and management of patients and caregivers with various health or disease conditions on the basis of modifiable risk and protective factors that span multiple conditions in order to address multiple co-morbidities and optimize outcomes in those co-morbidities. A system provides a platform with which one or more of medical histories, recent conditions, user information, and real-time measurement data of the patient and caregiver may be organized, tracked, and shared among one or more people who are involved in the caring of the patient to provide coaching (e.g., to one or more of the patient and caregiver) on modifiable factors. In addition to data sharing in a secured, private networking environment, the platform integrates essential functions for people in the various caregiver groups to communicate with a patient and each other in real-time so as to collaborate on the caring of the patient.

### TECHNICAL FIELD

The present disclosure generally relates to the field of digital healthcare, and, more particularly, to an electronic patient/caregiver advisor and healthcare system for the remote management of one or more chronic conditions.

### BACKGROUND

Conventional methods and systems for digital healthcare and management of chronic conditions are less than ideal in at least some respects. Although digital data can be acquired from patients in many ways, the integration of this digital data with patient treatment is less than ideal. For example, merely recording activity of a patient and suggesting an activity according to a predetermined treatment plan may not provide the best treatment for the patient. Accordingly, it is desirable to have a single knowledge-based application that can address fragmentation of care and lack of continuous tracking of patient health progress, and can provide personalized coaching towards a transformational lifestyle.

### SUMMARY

The present disclosure provides a system and method for the electronic monitoring and management of patients and caregivers with various health or disease conditions on the basis of modifiable risk factors that span multiple conditions in order to address multiple co-morbidities and optimize outcomes in those co-morbidities. The system described herein may provide a platform with which the medical histories, recent conditions, user information, and real-time measurement data for the patient may be organized, tracked, and shared among various people who are involved in the caring of the patient. In addition to data sharing in a secured, private networking environment, the platform may integrate essential functions for people in the various caregiver groups to communicate with a patient and each other in real-time so as to collaborate on the caring of the patient. The platform may enable patients and caregivers to share relevant data that may enhance treatment outcomes, such as location data (e.g., from tracking features). The platform may also provide care pathway services (e.g., refilling prescriptions and booking appointments), along with a diagnostic feature. On the basis of interactions (e.g., conversations) with both patients and caregivers, the system may include cognitive change screening in caregivers as well. The system may then help caregivers to address their own modifiable factors for dementia and may be a preventative tool to delay or slow down cognitive decline for not only the patient, but the caregivers as well.

The system for the diagnosis and management of one or more chronic health conditions may include a processor and a memory having programming instructions stored thereon. When executed by the processor, the programming instructions may cause the system to perform one or more operations. The system may register a patient and a caregiver via a single application. An electronic health record (EHR) for the patient may be retrieved from one or more repositories. Patient information may be received via a conversational user interface displayed on the patient's application. Health data may be received via one or more patient devices. Predictive models for actionable insights may be built for individual patients, groups of patients, and caregiver using one or more machine learning models and/or algorithms.

For example, a risk profile for the patient may be generated based on one or more of the EHR, the patient information, the relevant data from the patient, and information from the one or more repositories. One or more behavior change techniques may be generated based on the risk profile. The one or more behavior change techniques may be delivered to the patient as one or more of natural language messages via the conversational user interface.

### BRIEF DESCRIPTION OF THE DRAWINGS

The drawings described below are for illustration purposes only. The drawings are not intended to limit the scope of the present disclosure.

FIG. 1 is a diagram showing benefits of the disclosed platform in the treatment of chronic conditions;

FIG. 2 is a diagram illustrating the overall functioning of the platform;

FIG. 3 is a diagram illustrating how the platform can provide treatment and management of one or more chronic conditions on a single application;

FIG. 4 is a diagram illustrating how the platform allows patients and members of their caregiver team to coordinate in the treatment and management of dementia and/or mild cognitive impairment (MCI);

FIG. 5 is a diagram illustrating how the platform allows patients and members of their caregiver team to coordinate in the treatment and management of hypertension;

FIG. 6 is a diagram illustrating how the platform allows patients and members of their caregiver team to coordinate in the treatment and management of diabetes;

FIG. 7 is a diagram illustrating how the platform allows patients and members of their caregiver team to coordinate in the treatment and management of bipolar disorder;

FIG. 8 is a diagram illustrating a system diagram for the platform;

FIG. 9 is a diagram illustrating an exemplary one or more user devices;

FIG. 10 is a diagram illustrating hardware components of the one or more user devices;

FIG. 11 is a diagram illustrating the diagnostic and therapeutic components of the personalized medical system;

FIG. 12 is a flowchart showing an overall flow of patient treatment;

FIGS. 13A-13C are flowcharts showing a light version of the patient-facing application that includes in-application purchases; and

FIGS. 14A-14B are flowcharts showing a premium version of the application with all features unlocked.

### DETAILED DESCRIPTION

The present disclosure is related to digital healthcare, and, more particularly, to an electronic patient/caregiver advisor, and healthcare system for the remote management of one or more chronic conditions, which may be referred to as “myAVOS.” The myAVOS platform provides personalized coaching interventions through a conversational interface, which may be specifically adapted for elderly and/or cognitively impaired people. An accompanying application for caregivers, myAVOS CARE, may allow for the monitoring of both the patient and aspects of a caregiver's wellbeing, cognitive ability, and stress. This unique pairing provides information to and about the ecosystem surrounding someone with a chronic condition, such as dementia, with the aim to stabilize home care, predict emergencies, and screen the caregiver for any emerging concerns (e.g., early memory loss and burn out).

While competitors are emerging in this space, myAVOS is the only platform to integrate latest blockchain technology alongside artificial intelligence (AI) that allows for transactions (e.g., rewards and payments) between users (e.g., patients, caregivers, clinicians, academics, and providers).

The myAVOS platform is the only medical ecosystem combining professional healthcare knowledge with the swarm intelligence of patient communities, built-in medical and lifestyle data repositories and state-of-the-art behavioral change content utilizing blockchain to secure data and financial transactions and reward health-preserving behavior.

The following disclosure may include a platform and a single smartphone application for multiple indications of multiple chronic conditions. The platform may connect patients, caregivers, and physicians using patient-facing features and data analytics (e.g., sensor biometrics and digital biomarkers) to guide disease management, improve quality of life for patients and caregivers, and assists caregivers. The platform may be medication agnostic and may holistically and continuously assess both a patient's and caregiver's lifestyle and medical regimes. By optimizing both lifestyle and therapy compliance, the use of the disclosed platform may lead to improved clinical outcomes, a better quality of life for patients and caregivers, and significant cost savings.

The management of chronic conditions within aging populations across the world is rising in both cost and complexity. Traditional healthcare models cannot be sustained in terms of approach, resources, and scale. Aging populations drive huge increases in healthcare costs. According to research released by the Partnership to Fight Chronic Disease (PFCD) in April of 2016, 191 million people in the United States had at least one chronic condition in 2015. Further, 75 million Americans had two or more chronic conditions. Costs related to the management and treatment of chronic diseases from 2016 to 2030 is projected to be $42 trillion. Managing chronic conditions is a multi-dimensional problem. In the aging population (age>50), 65.2% of patients suffer from two or more comorbidities. The strong association of multimorbidity with age is well recognized yet more than half of people with multimorbidity and two-thirds with physical-mental health comorbidity are younger than 65 years old. According to the World Health Organization (WHO), around 50 million people have dementia worldwide, and the total number in 2050 will increase to 152 million. In addition, WHO estimates that the number of people with diabetes has risen from 108 million in 1980 to 422 million in 2014.

Conventional digital healthcare solutions are ineffective at managing chronic conditions. Despite multimorbidity being the norm rather than the exception, current clinical practice remains focused on managing single chronic diseases in isolation. In the U.S., patients spend in average 19 minutes per year with a physician. In Europe, it may take anywhere between 6-80 days to get an appointment with a doctor. Current solutions provide limited effect because they do not include patient preferences and social determinants, which are major factors in successful care outcomes. Digital healthcare platforms usually focus on resolving one condition at the time. Typically, the treatment of symptoms supersedes the understanding of the root cause of chronic medical issues. As described above, without addressing root cause of medical issues and the increase of multimorbidity associated with aging, the cost of long-term chronic conditions will reach $42 trillion by 2030.

In contrast, functional medicine focuses on determining how and why illness occurs and works on restoring health by addressing the root causes of disease for each individual. However, personalized treatment plans leading to improved patient outcomes require a detailed understanding of root cause including, for example: patient history and genetic information, biometrics information, the collection and tracking of biomarkers, and the inclusion of the patient's lifestyle factors, attitudes, and beliefs.

The myAVOS platform is meant to address the following questions: What would a dementia service look like if it were based on real data, shared by all parties involved? What if crises could be avoided by early warning triggers? What if the person with dementia and their family could manage their situation with personalised insights?

Dementia care pathways exist in many countries, and they offer a map of ideal services to help a person “live well” with dementia. However, most people don't experience a solid pathway at all, more likely a series of steppingstones, with the gaps in-between them sometimes difficult to traverse. This revolves around two factors: a dementia service is complicated and often involves different services, both statutory and voluntary, interwoven into a family's lives. Secondly, it is human nature to do the easy stuff first, so it is often the case that some services are provided by inadequate people.

From a specialist service perspective, dementia care arose out of the dedication and enthusiasm of early pioneers, especially in the 1970s, when older people with dementia were often housed in institutions. Old age psychiatry, geriatric psychiatry and geriatrics moved things swiftly forward, and memory clinics are now a key part of any specialist diagnostic service and were instrumental in overseeing the introduction of medication for Alzheimer's disease. They sit alongside multidisciplinary community services who offer speedy advice and help.

While much has been learned in research about what may cause dementia, the clinical practice has remained unchanged. In fact, the model now is very similar to other chronic disease conditions. Diabetes is often managed by a general practitioner (GP), with early specialist intervention if required and specialist nurse follow ups. Clearly having tangible measures like HbA1c to measure and monitor blood glucose levels in diabetic patients at home helps people feel more confident. The management of dementia is often similar, including early specialist diagnosis and specialist nurse follow ups, but patients tend to run out of much more expensive secondary care.

A success of specialist services has been to make people more aware of early symptoms, so that people are diagnosed much earlier than in the past. This has bought many benefits, but now people are coming forward with memory complaints, but no dementia. This is a group with a higher risk of developing dementia, for which no treatment is available. They have Mild Cognitive Impairment (MCI) or prodromal Alzheimer's disease (AD).

However, there is a body of research building around modifiable risk and protective factors for dementia, including exercise, diet, cognitive training, and aggressive treatment of cardiovascular risk factors. If an individual can reduce or delay their impairment, this must have an overall benefit. Much of this work is routine in primary care, so it should be managed from there. To involve specialist services only adds expense and detracts from the specialist work.

Dementia prevalence continues to rise. Using technology to collect individual data may improve communication, give personalised solutions, allow thresholds to be set for actions and help individuals manage their condition.

The following description includes a healthcare operating system that may be used by patients, caregivers and health care providers. The system may focus on resource and outcome optimization in multi-morbid patients and may combine medical and lifestyle interventions, pharmaceuticals, and smart technology with a functional whole-body approach that patients, regardless of location and mobility, may benefit from at the tip of their finger. The system may include a novel digital therapeutic platform that optimizes drug treatment and delivers cognitive behavioral interventions based on one or more of digital biomarkers (e.g., measurements of eye-tracking, micro-movements, gait, sleep patterns, typing variances), social determinants of health, nutritional analytics, and patient preferences. The platform may be interoperable with conventional electronic health record (EHR) and personal health record (PHR) systems and smart technology (e.g., wearables and mobile devices).

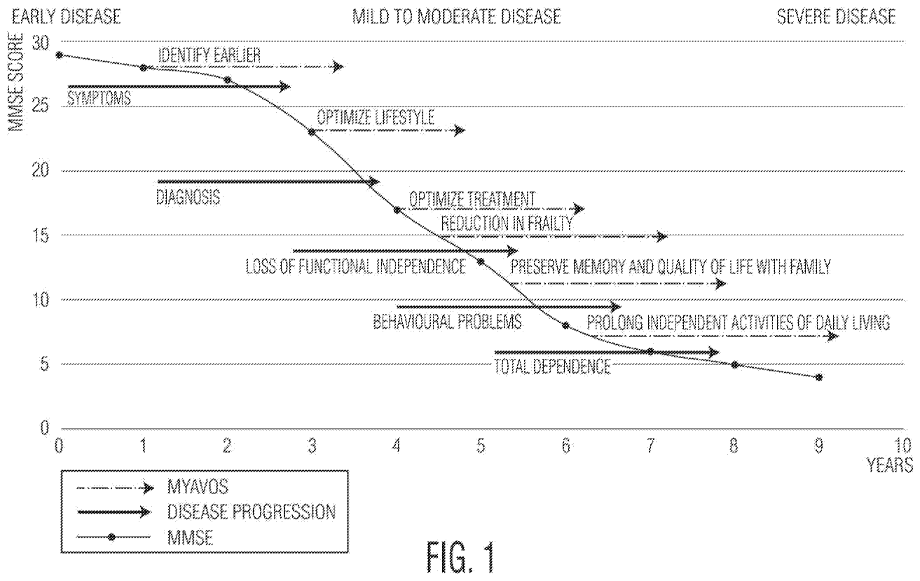

Referring now to FIG. 1, a diagram showing benefits of the platform in the treatment of chronic conditions is shown. The platform may provide a novel patient-centric approach to care, and a single application may be used to address fragmentation of care and lack of continuous tracking of patient health progress, and may provide personalized coaching towards a transformational lifestyle. Assessments and continuous coaching through a mobile application may provide patients with independence while extending their lifespan and improving their quality of life. The x-axis shows a number of years and the y-axis shows a scale for the Mini-Mental State Examination (MMSE). The line shows the MMSE score over time.

The platform may provide a number of novel improvements over conventional digital healthcare solutions, not only for patients and caregivers, but also to providers and payors (e.g., insurance companies, employee benefit programs, etc.). This may be accomplished by connecting better evidence with new and long-lasting health choices. For example, the platform may include a medication agnostic digital advisor that can uniquely optimize outcomes across multiple chronic conditions. The platform may create insights and actions through data, artificial intelligence (AI), and algorithms using one or more of the following: digital biomarkers, smart medical devices, mobile computing, sensor technologies; family, medical, and social history; patient activation management; patient reported outcomes; tailored behavioral therapies for lifestyle and treatment; tracking of treatment compliance; and disease progression tracked by real time measuring of biometric and digital biomarkers. The platform may facilitate needs along the chronic care pathway, such as scheduling, repeat prescription management, dispensing of emergency services, and geofencing and tracking.

For patients and caregivers the platform may provide one or more of: an improved patient and caregiver experience, a whole-body approach to condition management, personalized treatment plans, improved outcomes, portable medical information, and software that covers key medical needs. For providers, the platform may provide one or more of: augmented expertise in functional medicine, new treatment plans that seamlessly integrate current medical practices, time savings, analytical underpinnings to process large amounts of data to make it actionable, a single application that can cover over 50% of patients, and an improved patient and provider experience. For payers, the platform may provide one or more of: improved healthcare, technological innovations, increased efficiency, new real-world data (RWD) and real-world evidence (RWE), new touch points, and an improved member experience.

The platform may include one or more digital advisor user interfaces and/or applications (e.g., patient-specific and caregiver-specific) that are complemented with a comprehensive suite of capabilities that makes the application unique over conventional solutions. The single smartphone advisor for multiple chronic diseases is the most comprehensive platform for remote chronic disease management. The digital advisor application may be a single application that can manage multiple chronic indications and may include one or more of: a personalized virtual advisor, a symptom checker, a creation of a unique personal profile, integration of a nutrition repository, integration of a pharmacological database, an emergency dispatch service, and therapy agnostic management programs to improve medication non-adherence.

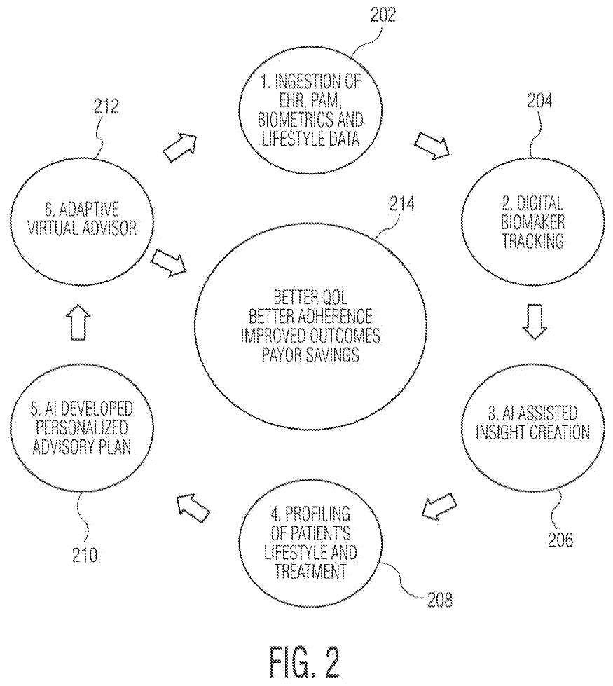

Referring now to FIG. 2, a diagram illustrating the overall functioning of the platform is shown. As shown in block 202, the platform may receive and manage relevant data from patients, caregivers, health care providers, and electronic sensors, including, for example, EHR information, patient activation measure information, biometric data, and lifestyle data. The platform may use nutritional analytics (e.g., as entered into a mobile application or captured via a camera), smartphone health kits, data from wearables and other smart devices, and geolocation as input data.

In block 204, the platform may track digital biomarkers (e.g., indicating brain health) of patients using one or more biometric devices and/or software programs. For example, in the treatment and management of dementia and/or mild cognitive impairment (MCI), the platform may track one or more of pulse, activity, location, gait, eye movement, assessments performed via mobile application, fall detection, and ambient temperature via one or more devices. In the treatment and management of bipolar disorder, the platform may track one or more of activity, pulse, location, sleep metrics, steps, continuous respiratory monitoring, social media posts, and a digital diary via one or more devices. In the treatment and management of hypertension, the platform may track blood pressure, activity, weight, and pulse via one or more devices. In the treatment and management of diabetes, the platform may track average blood glucose levels (hemoglobin A1c) levels, activity, weight, pulse, blood glucose levels, location, and fall detection via one or more devices.

In block 206, the platform may use artificial intelligence (AI) assisted insights to communicate with patients about, for example, symptoms, drug interactions and safety, and food/nutrition. In block 208, the platform may create and maintain a profile of the patient's lifestyle and treatment. In block 210, the platform may create AI driven personalized patient support programs to improve treatment adherence and self-management. In block 212, the platform may utilize an adaptive virtual advisor to engage patients. Block 214 illustrates the end results of the platform, which may be better quality of life for patients and caregivers, better adherence to treatment/management plans, improved outcomes, and payor savings. The platform may use a dynamic AI driven conversational user interface (UI) to provide health insights, coaching, brain training, and other services.

In combination, these features provide a novel approach to the successful screening, diagnosis, prevention, and management of chronic conditions in both patients and caregivers. Caregivers may be being screened early in life for cognitive change (e.g., decline) and may be coached to have a positive change in their modifiable factors. For patients with dementia and/or MCI, the platform may increase adherence rates, track cognitive function, slow cognitive decline, optimize treatment, improve and/or maintain independence, delaying the need for nursing home care, and facilitate the patient's long-term engagement with the digital advisor. For patients with bipolar disorder, the platform may predict relapses of the condition, intervene in the short term to prevent relapses, increase time between relapses and save costs for payors via reduced hospitalizations, and facilitate the patient's long-term engagement with the digital advisor. For patients with hypertension, the platform may increase medication adherence rates, contribute to a significant control of blood pressure, help patients achieve a better lifestyle as measured by activity and diet optimization, enable long-term risk reduction, and reduce medical expenditures. For patients with diabetes, the platform may increase patient usage and engagement, reduce the likelihood of out of range blood glucose readings, help patients achieve a better lifestyle as measured by activity and diet optimization, reduce medical expenditures, and reduce hospital and emergency room visits.

The platform may be cloud-based and secure for use on all conventional mobile operating systems (e.g., iOS and Android). The platform may be General Data Protection Regulation (GDPR) and Health Insurance Portability and Accountability Act (HIPAA) compliant and may utilize consent management and voice recognition. The platform may integrate real-world data and personal patient data in real time via, for example, a Fast Healthcare Interoperability Resources (FHIR) application programming interface (API) that connects to patient EHR, API connectivity to third party repositories and/or AI engines, and an open architecture connectivity to wireless devices. The platform may use embedded analytics, including, for example, passive medication adherence detection, AI driven user stratification, and AI driven personalized coaching. The platform may provide clinical decision support and/or intervention using one or more of AI assessment of treatment progression, treatment optimization through telemedicine, and virtual coaching (e.g., through one or more of a conversational user interface, SMS, notifications, telephone, or in-person caregivers). The platform is scalable and able to incorporate multiple languages, new indications, and technologies. The platform UI may also provide customized dashboards for all members of the caregiving team, including enrolling clinicians, patients, caregivers, and health care providers.

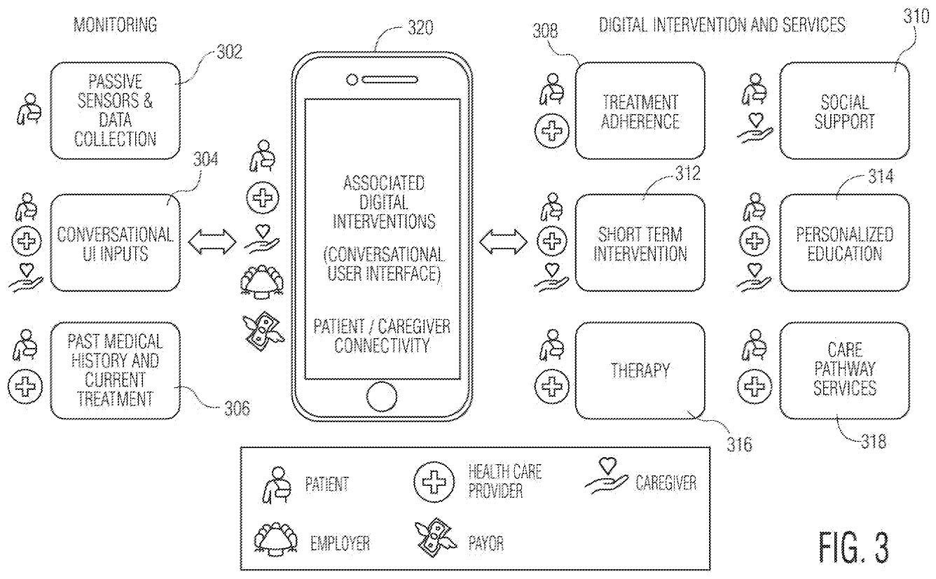

Referring now to FIG. 3, a diagram illustrating how the platform can provide treatment and maintenance of one or more chronic conditions on a single application is shown. As shown in the diagram, the platform allows patients, employers, health care providers, caregivers, and payors to coordinate and collaborate in different aspects of a treatment plan. It should be noted that patients may be involved in each aspect of the treatment program.

For example, as shown in block 302, during monitoring, information from the patient may be collected by passive sensors and other data collection. As shown in block 304, the patient, health care provider, and caregiver may use conversational user interface (UI) inputs. As shown in block 306, the patient and healthcare provider may provide past medical history and current treatment. The platform may provide one or more digital intervention services. For example, as shown in block 308, the patient and healthcare provider may coordinate for treatment adherence. In block 310, the patient and caregiver may coordinate for social support. In block 312, the patient, health care provider, and caregiver may coordinate for short term intervention. In block 314, the patient, health care provider, and caregiver may coordinate for personalized education. In block 316, the patient and health care provider may coordinate for therapy. In block 318, the patient and health care provider may coordinate for care pathway services. As shown in block 320, the patient, health care provider, caregiver, employer, and payor may all coordinate using the myAVOS platform. In an example, the myAVOS platform may provide a patient with a specific user interface and/or application and a caregiver with a different user interface and/or application.

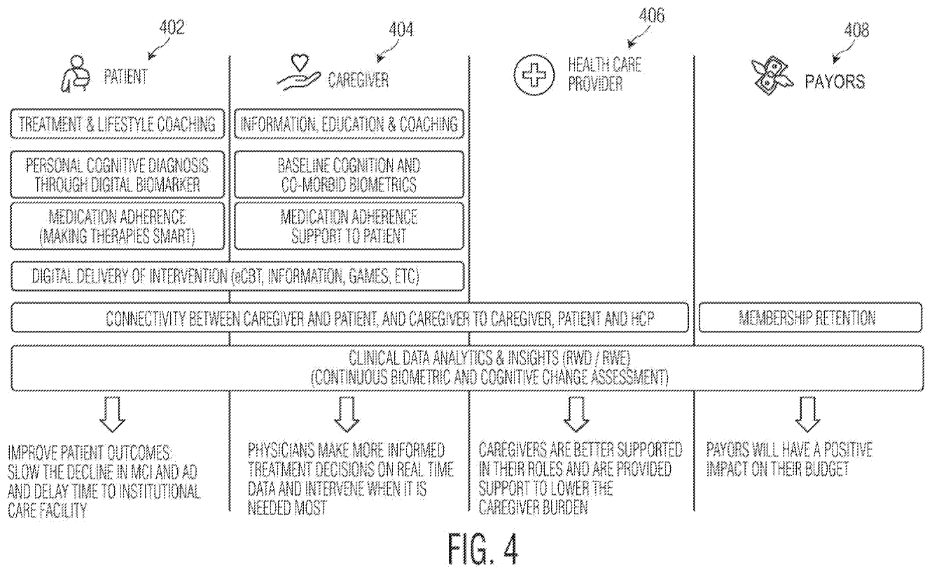

Referring now to FIG. 4, a diagram illustrating how the platform allows a patient 402 and members of the patient's caregiver team (e.g., a caregiver 404, a physician 406, and a payor 408) to coordinate in the treatment and management of dementia and/or mild cognitive impairment (MCI). Using passive sensors and/or data collection, the platform may interact with the patient through a conversational user interface (e.g., a conversational user interface, an avatar, etc.) and track the patient's sleep, location, motion, eye movements, and cognition. Alerts may be generated and delivered to one or more of the patient, a caregiver, and a health care provider based on one or more of geofencing and vital signs. Medication adherence may be tracked and short-term interventions and/or therapy may be provided to patients through the mobile application. The platform may provide social networking features to patients for community support and personalized education materials. The platform may be able to not only manage multiple diseases, but may also address modifiable risk factors with evidence-based suggestions that offer guidance for the mitigation of dementia and/or MCI and enable better outcomes in these diseases.

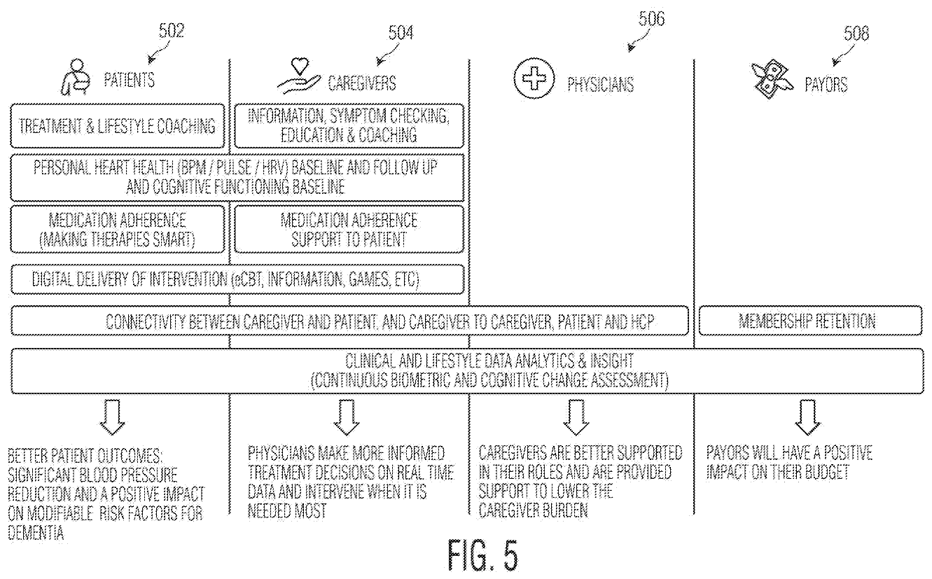

Referring now to FIG. 5, a diagram illustrating how the platform allows a patient 502 and members of the patient's caregiver team (e.g., a caregiver 504, a physician 506, and a payor 508) to coordinate in the treatment and management of hypertension. Using passive sensors and/or data collection, the platform may interact with the patient through a conversational user interface and track the patient's blood pressure, steps, weight, pulse, and diet. Alerts may be generated and delivered to one or more of the patient, a caregiver, and a health care provider based on one or more vital signs. Medication adherence may be tracked and short-term interventions and/or therapy may be provided to patients through the mobile application. The platform may provide social networking features to patients for community support and personalized education materials. The platform may be able to not only manage multiple diseases, but may also address modifiable risk factors with evidence-based suggestions that offer guidance for the mitigation of hypertension and enable better outcomes in these diseases.

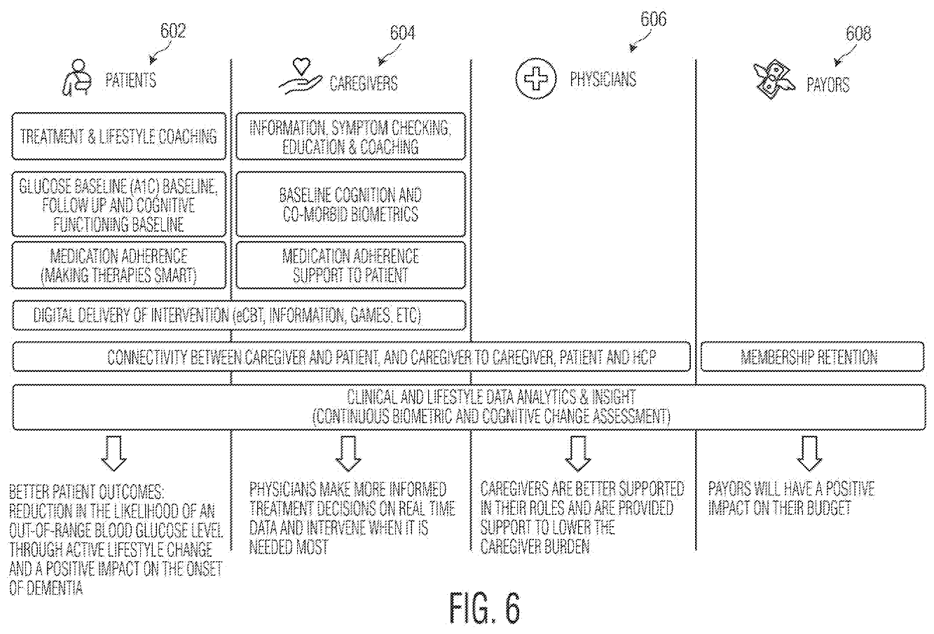

Referring now to FIG. 6, a diagram illustrating how the platform allows a patient 602 and members of the patient's caregiver team (e.g., a caregiver 604, a physician 606, and a payor 608) to coordinate in the treatment and management of diabetes. Using passive sensors and/or data collection, the platform may interact with the patient through a conversational user interface and track the patient's blood pressure, steps, weight, pulse, and diet. Alerts may be generated and delivered to one or more of the patient, a caregiver, and a health care provider based on one or more vital signs. Medication adherence may be tracked and short-term interventions and/or therapy may be provided to patients through the mobile application. The platform may provide social networking features to patients for community support and personalized education materials. The platform may be able to not only manage multiple diseases, but may also address modifiable risk factors with evidence-based suggestions that offer guidance for the prevention of diabetes and enable better outcomes in these diseases.

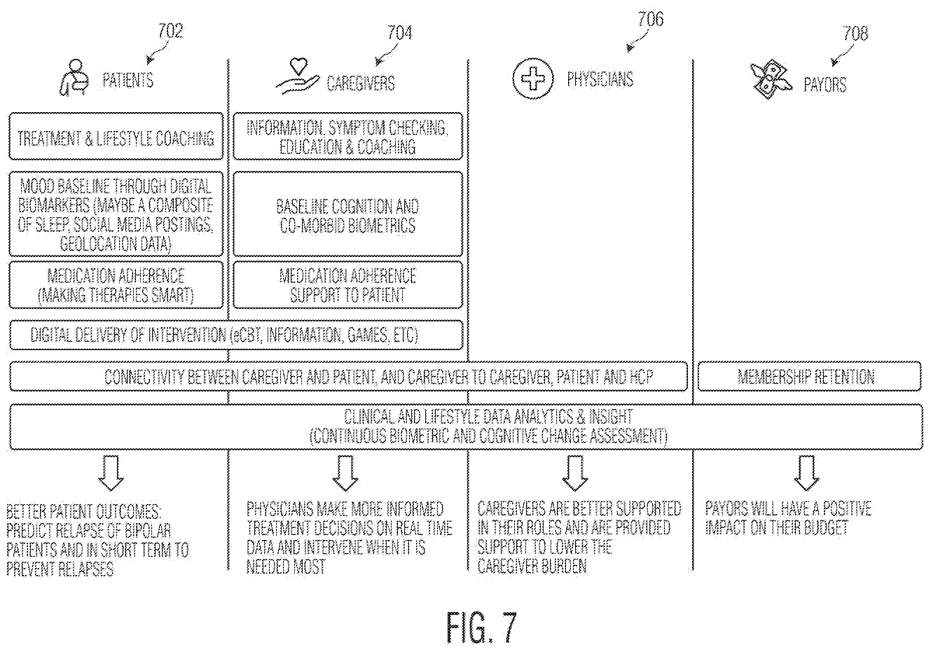

Referring now to FIG. 7, a diagram illustrating how the platform allows a patient 702 and members of the patient's caregiver team (e.g., a caregiver 704, a physician 706, and a payor 708) to coordinate in the treatment and management of bipolar disorder. Using passive sensors and/or data collection, the platform may interact with the patient through a mood diary and track the patient's sleep, location, steps, biometrics, and social media posts. Alerts may be generated and delivered to one or more of the patient, a caregiver, and a health care provider based on one or more of geofencing and vital signs. Medication adherence may be tracked and short-term interventions and/or therapy may be provided to patients through the mobile application. The platform may provide social networking features to patients for community support and personalized education materials. The platform may be able to not only manage multiple diseases, but may also address modifiable risk factors with evidence based suggestions that offer guidance for the prevention of bipolar disorder and enable better outcomes in these diseases.

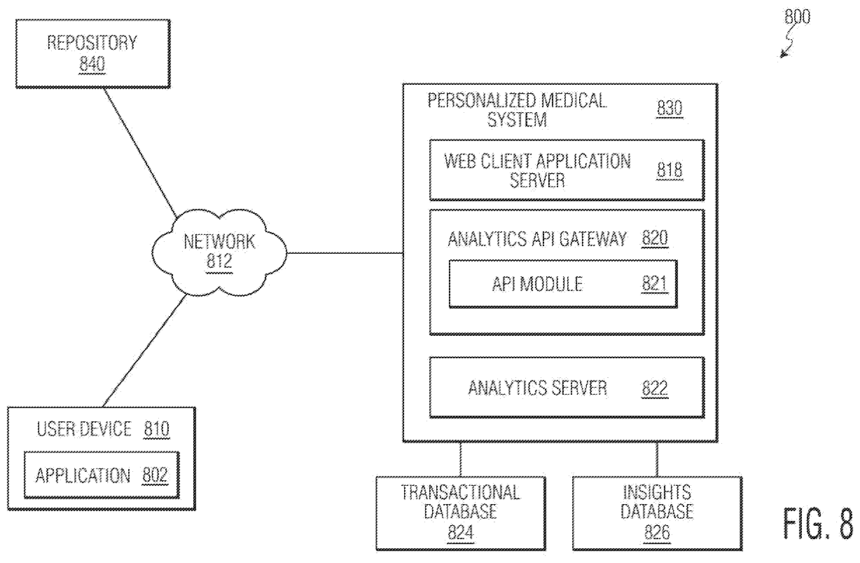

Referring now to FIG. 8, a system diagram illustrating a platform 800 is shown. The platform 800 may provide diagnosis and treatment of one or more chronic conditions through a single application 802 on one or more user devices 810 of one or more of patients, caregivers, health care providers, and payors. The one or more user devices 810 may include, for example, one or more of a mobile device such as a smart phone, an activity monitor, or a wearable digital monitor, may record data, metadata, and biometrics related to a patient. The data may be collected based on interactions of the patient with the user device, as well as based on interactions with caregivers and health care professionals. The data may be collected actively, such as by interactions with AI conversational user interfaces and/or caregivers, recording speech and/or video, and recording responses to diagnostic questions. The data may also be collected passively, such as by the activity monitor or by monitoring online behavior and social media of patients and caregivers.

The one or more user devices 810 may be connected to a computer network 812, allowing it to share data with and receive data from connected computers. In particular, the one or more user devices 810 may communicate with a personalized medical system 830, which may include a server configured to communicate with the one or more user devices 810 over the computer network 812. The computer network 812 may be of any suitable type, including individual connections via the Internet, such as cellular or Wi-Fi networks. In some embodiments, network 105 may connect terminals, services, and mobile devices using direct connections, such as radio frequency identification (RFID), near-field communication (NFC), Bluetooth™, low-energy Bluetooth™ (BLE), Wi-Fi™, ZigBee™, ambient backscatter communication (ABC) protocols, USB, WAN, or LAN. Because the information transmitted may be personal or confidential, security concerns may dictate one or more of these types of connection be encrypted or otherwise secured. In some embodiments, however, the information being transmitted may be less personal, and therefore, the network connections may be selected for convenience over security.

The computer network 812 may include any type of computer networking arrangement used to exchange data or information. For example, the computer network 812 may be the Internet, a private data network, virtual private network using a public network and/or any other suitable connection that enables components in the platform 800 to send and receive information between the components of the platform 800.

The personalized medical system 830 may include a processor, a memory, a storage, and a network interface. The processor may retrieve and execute program code (i.e., programming instructions) stored in the memory, as well as store and retrieve application data. The processor may be one or more of a single processor, multiple processors, a single processor having multiple processing cores, and the like. The network interface may be any type of network communications the personalized medical system 830 may utilize to communicate externally via the network 812.

The storage may be, for example, a disk storage device. The storage may be a combination of fixed and/or removable storage devices, such as fixed disk drives, removable memory cards, optical storage, network attached storage (NAS), storage area network (SAN), and the like.

The memory may include one or more of an operating system and program code. The program code may be accessed by the processor for processing (i.e., executing program instructions). The program code 566 may include, for example, executable instructions configured to perform one or more operations described herein.

The personalized medical system 830 may represent a computing platform configured to host a plurality of servers. In some embodiments, the personalized medical system 830 may be composed of several computing devices. For example, each computing device of the personalized medical system 830 may serve as a host for a cloud computing architecture, virtual machine, container, and the like.

The personalized medical system 830 may include at least a web client application server 818, an analytics application programming interface (API) gateway 820, and an analytics server 822. The web client application server 818 may be configured to host the application 802 and/or one or more webpages that allow the one or more user devices 810 to access the platform.

The analytics server 822 may be configured to generate one or more actionable insights based on user activity data. For example, the analytics server 822 may consume high-resolution data generated by the one or more user devices 810. From the high-resolution data, the analytics server 822 may be configured to generate one or more actionable insights related to a patient's health. It should be noted that the analytics server 822 may be composed of several computing devices. For example, each computing device of the analytics server 822 may serve as a host for a cloud computing architecture, virtual machine, container, and the like.

The analytics API gateway 820 may be configured to act as an interface between the web client application server 818 and the analytics server 822. For example, the analytics API gateway server 820 may be configured to transmit data collected by web client application server 818 to the analytics server 822. Likewise, the analytics API gateway server 820 may be configured to transmit insights generated by the analytics server 822 to the web client application server 818.

The analytics API gateway 820 may include an API module 821. The API module 821 may include one or more instructions to execute one or more APIs that provide various functionalities related to the operations of the personalized medical system 830. The API module 821 may include an API adapter that allows API module 821 to interface with and utilize APIs maintained by the personalized medical system 830 and/or an associated entity. In some embodiments, APIs may enable the personalized medical system 830 to communicate with the one or more user devices 810 and/or a repository 840. The repository 840 may include a computer system storing one or more of, for example, EHR, nutritional databases, pharmacological databases, symptom diagnosis databases, biomarker databases, and therapeutic databases.

The personalized medical system 830 may communicate with a transactional database 824 and an insights database 826. The transactional database 124 may be configured to store raw data received (or retrieved) from the one or more third party devices 810 and/or the repository 840. For example, the web client application server 818 may be configured to receive data from the one or more third party devices 810 and/or the repository 840 and store that data in the transactional database 824. The insights database 826 may be configured to store one or more actionable insights generated by the analytics server 822. In operation, for example, the analytics API gateway 820 may pull information from the transactional database 824 and provide the information to the analytics server 822 for further analysis. Upon generating one or more actionable insights via artificial intelligence and machine learning algorithms, the analytics server 122 may be configured to store the generated insights in the insights database 826. The analytics API gateway 120 may, in turn, pull the generated insights from the insights database 826 and provide the insights to the web client application server 818 for transmission to the one or more third party devices 810 and/or the repository 840.

Information related to diagnosis and therapy may also be exchanged between the personalized medical system 830 and the repository 840. An example of a repository 840 may be a healthcare gateway to provide EHR/PHR information. The healthcare gateway may provide structured data and an advanced audit trail including one or more of: a name of the clinician who has viewed the patient's record, the viewing organization, date and time the record was accessed, the viewing organization department, consent status, and a sharing agreement. The healthcare gateway may provide one or more of a summary (e.g., current problems, current medication, allergies, and recent tests), a problem view, a diagnosis view, a medication view (e.g., current, past, and issues), risks and warnings, procedures, investigations, examination (e.g., blood pressure), events (e.g., encounters, admissions, and referrals), and patient demographics. The service may provide one or more of the following: timely patient information to clinicians to enable improvements to patient care, a reduction in duplication of effort and errors in patient management, improved information sharing, improvements in service communication and information sharing over existing paper based communications, and reduction in administrative overheads.

Another example of a repository 840 may be a patient support management content system configured to deliver configurable patient support programs for clients. The system may receive anonymized patient and enrolment details from the application 802 and pass content back to the application 802, which may then decide what happens with the content (e.g., fulfill internally and/or share with patient using the conversational user interface).

Another example of a repository 840 may be a comprehensive, accessible, online database containing information on drugs and drug targets. For example, the repository 840 may be a bioinformatics and a cheminformatics resource that combines detailed drug (i.e. chemical, pharmacological and pharmaceutical) data with comprehensive drug target (i.e. sequence, structure, and pathway) information. The repository 840 may have several endpoints to support different types of searches and filtering. The repository 840 may utilize one or more databases, which may be updated daily by a team of biochemists, pharmacists, and scientists.

Another example of a repository 840 may be a food and nutrition database that provides nutritional information. In an example, a user may enter food information manually, may search for items in a database, or may use a camera to capture a barcode or a QR code.

Another example of a repository 840 may include an API for user triage and preliminary medical diagnosis that can help implement an intelligent symptom checker or an adaptive intake form. A user's health data (e.g., symptoms, risk factors, lab tests results or demographics) may be sent to the repository 840 and an inference engine may analyze the data and provide a list of likely conditions and relevant observations to verify. The personalized medical system 830 may assess whether or not symptoms reported by a user are a result of medication, which may inform attitudes and beliefs of users and lead to treatment non-adherence. This may be included on a user risk profile to trigger the right cognitive behavior therapy content.

Another example of a repository 840 may be provide a survey that assesses a person's underlying knowledge, skills and confidence integral to managing his or her own health and healthcare. The survey may cover a number of relevant domains (e.g., daily activities, medications, emotions, symptoms, and social interactions).

Another example of a repository 840 may monitor cognition by evaluating perceptual and memory function in individuals at risk or with mild cognitive impairment/prodromal Alzheimer's disease. In an example, the repository 840 may use machine learning (ML) to analyze digital biomarkers and may generate a detailed report of features organized per cognitive domain for easy translation into everyday practice (e.g., disease related outcomes and clinical trial endpoints). The repository 840 may objectively assess cognitive and everyday functions, in the most ecologically valid way, prior to the onset of symptoms. The repository 840 may leverage AI, ML, and augmented reality (AR) to detect early & subtle micro-errors (accuracy) and micro-movements (latency) during everyday function, up to 10 years prior to the onset of typical neurodegenerative symptoms. The repository 840 may utilize digital biomarkers to detect brain changes early in the disease process, helping to accelerate early diagnosis for better clinical outcomes. The repository 840 may utilize voice enabled machine learning tools to screen and monitor for cognitive change. Screening may be combined with intervention in one application for both patients (i.e., therapeutic care) and caregivers (preventive care).

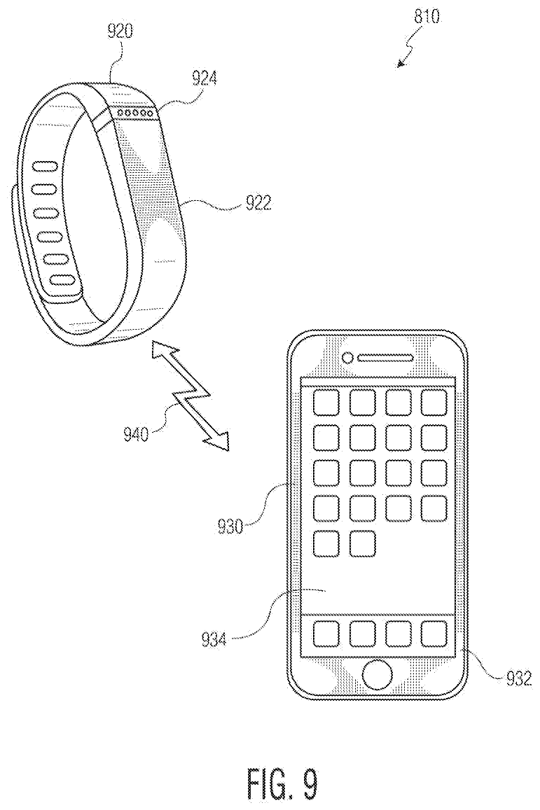

Referring now to FIG. 9, a diagram illustrating an exemplary one or more user devices 810 is shown. In an example, the one or more user devices 810 may include at least one health monitoring device 920 and an electronic display device 930 in communication therewith. The health monitoring device 920 may be designed and dimensioned to be worn on or carried by the body of a user and may collect activity information or other health information about the user. In another example, the health monitoring device 920 may comprise a non-portable device configured to measure one or more health parameters of the user. For example, the health monitoring device 920 may measure the user's weight and/or nutrition. Moreover, an individual user may be associated with more than one health monitoring device 920.

The health monitoring device 920 may be in communication with the electronic display device 930, and may be configured to deliver collected data about the user to the electronic display device 930. The collected data may include activity data (e.g., measured in distance, steps, flights of stairs, etc.) or other health data. Examples of the collected data may include sleep data, nutrition data, weight data, heart rate data, location data, and other environmental data (including temperature, humidity, precipitation, altitude, etc.). The electronic display device 930 may process the data and display it to the user and/or may transmit the data to the personalized medical system 830.

The health monitoring device 920 may be provided in any of various forms and is configured to collect any of various types of health data related to a user. Such data may be, for example, blood glucose levels, blood oxygen levels, heart rate, and human kinematic and/or physiological data that provides personal metrics information about a level of activity or type of activity during awake times, and sleep quality, amount, and/or other sleep information during sleep times. Accordingly, the health monitoring device 920 may be configured to collect one or more of step data, body motion data, distance traversal data, altitude data, heart rate data, body temperature data, breathing data, environmental/positional data (such that provided by a GPS receiver), food consumption data, or any of various other types of personal or environmental metrics that may be relevant to determining health parameters including awake time activities and/or sleep quantity and quality of the user.

The term “health data” as used herein refers to data associated with the user during the user's wake time or sleep time, and such data may indicate the user's participation in any of various activities including eating, sleeping, high intensity activity, sedentary activity, and various degrees of activity in-between. Examples of health data include step data, body motion data, distance traversal data, altitude data, heart rate data, body temperature data, breathing data, environmental/positional data (such that provided by a GPS receiver), food consumption data, weight and/or body fat data, or any of various other types of personal metrics that may be relevant the user's health.

The term “activity data” as used herein is a subset of health data, and refers to data related to physical activity (i.e., movement or lack thereof) of the user. Examples of activity data include step data, body motion data, distance traversal data, altitude data, heart rate data, breathing data, environmental/positional data (such that provided by a GPS receiver), or any of various other types of personal activity metrics that may be relevant the user's physical activity for a given period of time.

Health data may be collected via manual entry by the user, automatically by a sensor of the health monitoring device 920, and/or collected by any of various other means. The term “personal metric” as used herein refers to any of various measures of health data that may be defined by any of various parameters (e.g., user heart rate expressed as beats per minute, user activity defined by total steps for a day, distance traversed for some time period, calories spent, calories consumed, total time of activity, body weight, amount of body fat, sleep quality defined by sleep time and/or sleep quality/sleep cycles, any of the foregoing expressed as a percentage of a goal or other standard, etc.). In an example, the health monitoring device 920 may be an activity tracker configured to measure one or more of steps taken (including walking or running), distance traversed, stairs climbed, heart rate, as well as various other personal metrics (such “activity trackers” are commonly also referred to as “fitness trackers”). These activity trackers may further process the measured parameter to determine other personal metrics such as calories spent, sleep quality, etc. Such further processing may occur on the activity tracker itself or in association with other computer devices in communication with the activity tracker. Additional or alternative examples of health-monitoring devices 920 include those sold under the trademarks FITBIT®, JAWBONE®, POLAR®, APPLE®, UNDER ARMOUR®, OMRON® and GARMIN®.

In an example, the health monitoring device 920 may be configured to be worn or carried by the human user. The health monitoring device 920 shown in FIG. 9 may be a wrist band that the user straps to his or her wrist. However, it will be recognized that in other embodiments, the health monitoring device 920 may be provided in any of various different configurations to be worn on any of various locations on the body of the user, such as via a module that clips on to clothing, is worn on a chest strap, fits in a pocket of the user, and/or is incorporated into a garment or a shoe. Alternatively, the health monitoring device 920 may be fixed and non-portable device (i.e., not worn by the user), such as for example, a so-called smart scale onto which a user stands and/or a tablet or personal computing device into which the user enters health-related data, such as nutritional data. Additional examples of configurations for the health monitoring device 920 include configurations where the sensor device is provided as a component of a multi-function device, such as a watch, a mobile phone or other personal electronic device.

In FIG. 9, the health monitoring device 920 is shown as being a completely separate unit from the display device 930. However, the health monitoring device 920 and the display device 930 may be provided as a single unit. For example, the health monitoring device 920 and the display device 930 may be provided as part of a mobile phone, so-called “smart” watch, or other personal electronic device. While a single health monitoring device 920 is shown in FIG. 1, it will be recognized that multiple sensor devices may be used by a single user, each of the health monitoring device 920 configured for communication with the electronic display device 930.

The health monitoring device 920 may include a housing 922 and an I/O interface 925, which may include a display 924, one or more connection ports (not shown), or other input and output hardware and software. The display 924 may vary based on the type of device. For example, the display 924 may simply be one or more colored lights and/or flashing patterns configured to communicate information to the user (e.g., progress towards a goal or other personal metric). In another example, the display 924 may be an LCD or LED screen that provides more specific personal metric information to the user (e.g., total number of steps for the day, progress towards a goal, heart rate, some combination thereof, etc.). The connection ports may be used to connect the health monitoring device 920 to a power source or to share data with other electronic devices.

The display device 930 may include a protective outer shell or housing 32 designed to retain and protect the electronic components positioned within the housing 932. The housing 932 may comprise any number of shapes, configurations, and/or materials, the description herein being merely exemplary. In an example wherein the display device 930 also functions as the one or more health monitoring devices 920, the housing 932 may serve as a common housing for components of the display device 930 and components of the health monitoring device 920. The display device 930 may include a display screen 934 configured to visually display graphics, text and other data to the user. The display screen 934 may be a touch screen display that allows the user to see data presented on the display screen 934 and input data into the display device 930 via a keyboard on the touch screen. The display device 930 may also include a microphone and/or speakers to facilitate audio communications with the user and/or verbal entry of commands to the display device 930.

It will be recognized that the health monitoring device 920 and the display device 930 may communicate via, for example, one or more of a mobile telephony network, the Internet, and/or a global positioning system (GPS).

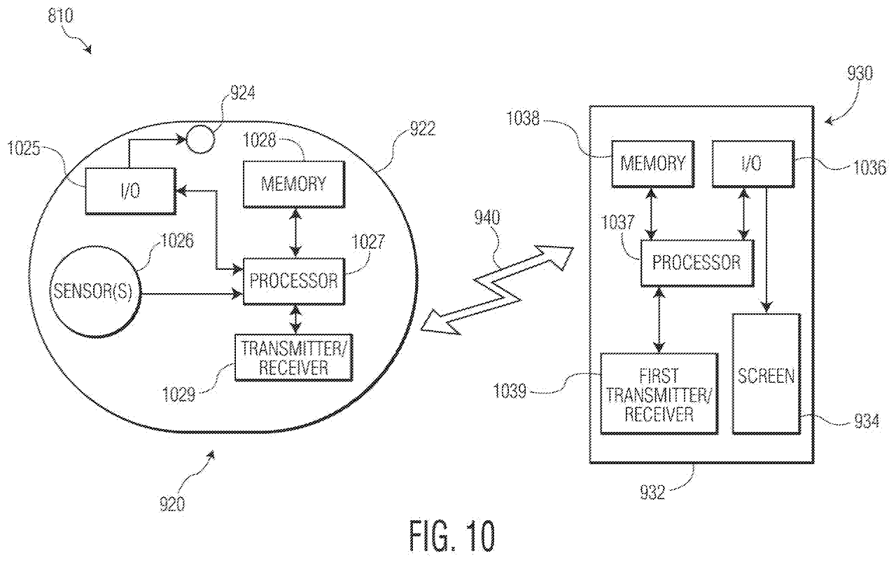

Referring now to FIG. 10, a diagram illustrating hardware components of the one or more user devices 810 that may be used by a user is shown. The health monitoring device 920 may include electronic circuitry comprising one or more sensors 1026 (optional), a processor 1027, a memory 1028, and a transceiver 1029. The health monitoring device 920 may also include a battery or other power source (not shown) configured to power the various electronic devices within the health monitoring device 920. The battery of the health monitoring device 920 may be a rechargeable battery. In this example, the health monitoring device 920 may be placed in or connected to a battery charger in order to recharge the battery.

The health monitoring device 920 may include one or more sensors 1026. The sensors 1026 may include any of various devices configured to collect the activity data, including step data, motion data, distance traversal data, GPS data, body weight data, altitude data, heart rate data, body temperature data, breathing data, environmental/positional data, or any of various other types of personal metrics that may be relevant to determining activities and biometrics of the wearer. The sensors 1026 may include a 3-axis accelerometer configured to detect the steps of the wearer during walking and running, and general movements of the wearer during more sedentary periods such as sleep. Of course, it will be recognized by those of ordinary skill in the art that numerous other sensors may be used, depending on the type of activity and biometrics the health monitoring device 920 is designed to detect.

The processor 1027 may be any of various microprocessors as will be recognized by those of ordinary skill in the art. The processor 1027 may be configured to receive data signals from the sensors 1026 and other component parts of the health monitoring device 920 (such as data entered via an I/O input 1025) and process such signals. The processor 1027 may be connected to the memory 1028 and the transceiver 1029, and may deliver processed data to one or both of the memory 1028 and the transceiver 1029. Additionally, the processor 1027 may perform some processing on the received data prior to delivery thereof to the memory 1028 or transceiver 1029. For example, the processor 1027 may associate the data with a particular time, day, user (in the instance that the device is configured to collect data relating to more than one user), and/or event. The processor 1027 may also be connected to the I/O interface 25, and may send signals to the I/O interface 1025 which results in illumination of the display 924 in order to provide text and/or image based messages or otherwise communicate to the user.

The memory 1028 may be configured to store information, including both data and instructions. The data generally includes, e.g., health data, activity data, health-parameter data, etc. that may be retrieved from the processor 1027. The instructions stored at the memory 1028 generally include firmware and/or software for execution by the processor 1027, such as a program that controls settings, a program that controls the output of the display 924 on the health monitoring device 920, a program that controls the receipt of information via the sensor 1026, a program that controls the transmission and reception of data via the transceiver 1029, as well as any of various other programs that may be associated with the health monitoring device 920. Such instructions may be present on the device 920 at the time of manufacture or may be downloaded thereto via well-known mechanisms.

The memory 1028 may be of any type capable of storing information accessible by the processor 1027, such as a memory card, ROM, RAM, write-capable, read-only memories, or other computer-readable medium. The data may be stored in the memory 1028 in a relational database as a table having a plurality of different fields and records, XML documents, or flat files. The data may also be formatted in any computer-readable format such as, but not limited to, binary values, ASCII or Unicode.

The transceiver 1029 may include an radio frequency (RF) transmitter and receiver configured to transmit and receive communications signals over a short range using a wireless communications technology, such as Bluetooth®, using any of various communications protocols, such as TCP/IP. Such transceivers are well known and will be recognized by those of ordinary skill in the art. The transceiver 1029 may be configured to communicate with the display device 930 when the health monitoring device 920 is within a given range of the display device 930, and transmit activity data to the display device 930.

While the health monitoring device 920 has been described herein as the primary device for collecting and transmitting health parameter data to the display device 930, it will be recognized that additional data may also be collected or otherwise obtained and/or input in to the display device 930 via various other mechanisms. The user may manually input data into the health monitoring device 920 and/or the display device 930.

The display device 930 may be a handheld computing device, such as a smartphone. The display device 930 generally includes an input/output interface 1036, a processor 1037, a memory 1038, and a transceiver 1039. While a smartphone has been shown as the display device 930 in FIG. 9, it will be appreciated that the display device 930 may alternatively comprise any number of alternative devices. For example, the display device 930 may be a standalone device, such as a desktop PC or smart television. Alternatively, the display device 930 may be any type of portable or other personal electronic device such as a watch, tablet computer, laptop computer, or any of various other mobile computing devices. As will be recognized by those of ordinary skill in the art, the components of the display device 930 may vary depending on the type of display device used. Such alternative display devices may include much (but not necessarily all) of the same functionality and components as the display device 930 shown in FIG. 10, as well as additional functionality or components necessary for proper functioning thereof (not shown). In addition, the display device 930 may function as one of the one or more health monitoring devices 920 discussed elsewhere herein.

The processor 1037 of the display device 930 may be any of various processors as will be recognized by those of ordinary skill in the art. The processor 1037 may be connected to the I/O interface 1036, the memory 1038, and the transceiver 1039, and may be configured to deliver data to and/or receive data from each of these components. The processor 1037 may be configured to process raw health-parameter data received from the one or more health monitoring devices 920 and transmit the data to the personalized medical system 830 and/or transform the data into a graphical format for presentation on the display screen 934. It will be recognized by those of ordinary skill in the art that a “processor” as used herein includes any hardware system, hardware mechanism or hardware component that processes data, signals or other information. A processor may include a system with a central processing unit, multiple processing units, dedicated circuitry for achieving functionality, or other systems.

The I/O interface 36 of the display device 30 includes software and hardware configured to facilitate communications with the one or more health monitoring devices 920 and/or communications to the user him/herself.

The memory 1038 may be configured to store information, including both data and instructions. The data may be, for example, health-parameter data as discussed above, which may be related to the activities, nutrition, sleep, environment, etc. of the user, along with other data that may be ancillary to the basic operation of the display device and any applications retained on the display device. The instructions stored at the memory 1038 generally include firmware and other software for execution by the processor 1037, such as a program that controls the settings for the display device, a program that controls the output of the display 934 on the display device 930, programs that control various applications on the display device, a program that controls the transmission and reception of data via the transceiver 1039, as well as any of various other programs that may be associated with the display device 930. As explained in further detail below, the instructions stored in the memory 1038 for execution by the processor may include, for example, an activity or health tracking app, a health perception tool, and/or a nutrition estimate tool.

The memory 1038 may be of any type of device capable of storing information accessible by the processor, such as a memory card, ROM, RAM, write-capable memories, read-only memories, hard drives, discs, flash memory, or any of various other computer-readable medium serving as data storage devices as will be recognized by those of ordinary skill in the art.

Portions of the system and methods described herein may be implemented in suitable software code that may reside within the memory. Such software code may be present on the display device 930 at the time of manufacture or may be downloaded thereto via well-known mechanisms. A computer program product implementing an embodiment disclosed herein may therefore comprise one or more computer-readable storage media storing computer instructions translatable by a processor to provide an embodiment of a system or perform an embodiment of a method disclosed herein. Computer instructions may be provided by lines of code in any of various languages as will be recognized by those of ordinary skill in the art. A “computer-readable medium” may be any type of data storage medium that can store computer instructions, including, but not limited to the memory devices discussed above.

The transceiver 1039 may be an RF transmitter and receiver configured to transmit and receive communications signals over a short range using a wireless communications technology, such as Bluetooth®, using any of various communications protocols, such as TCP/IP. Such transceivers are well known and will be recognized by those of ordinary skill in the art. The transceiver 1039 may be configured to communicate with the transceiver 1029 of the health monitoring device 920. The display device 930 may also include a battery or other power source (not shown) configured to power the transceiver 1039 and various other the electronic components within the display device 930. The transceiver 1039 may be configured to allow the display device 930 to communicate with a wireless telephony network, as will be recognized by those of ordinary skill in the art. The wireless telephony network may comprise any of several known or future network types. For example, the wireless telephony network may comprise commonly used cellular phone networks using CDMA or FDMA communications schemes. Some other examples of currently known wireless telephony networks include Wi-Fi, Wi-Max, GSM networks, as well as various other current or future wireless telecommunications arrangements.

Raw health data collected by the health monitoring device 920 may be processed by the display device 930 and/or delivered to the personalized medical system 830 for further processing. The processing to be performed may depend on various factors including the type of data received and different subscriptions of the user. Examples of such processing are provided in the paragraphs below.

Typical processing may relate to the user's current activity level, trends, history, etc. For example, the one or more computers that processes the raw data may calculate an activity level which may be based on a combination of inputs, including, for example, steps taken over a period of time, heart rate, etc. In another example, GPS data is used to determine location of the user, total distance travelled, or the route taken by the user during a period of time.

Furthermore, the health data may be processed into different forms and formats, depending on the particular device that will ultimately be used to view the data. For example, the data may be processed into a first format that will allow it to be viewed on e.g., a smart watch and into a second format that will allow it to be viewed on the monitor of a personal computer; that is a compressed or summarized format for the smaller display and a more detailed format for the larger and more powerful display.

In the instance a user carries the one or more health monitoring devices 920, health data may be delivered to the display device 930. As represented by arrow 940, the one or more health monitoring devices 920 may be configured to transmit a wireless RF signal representative of the health data collected or obtained to at least one display device 930. In addition, the health data may also be transmitted to additional computing devices (display devices 930), such as a watch or a laptop computer where the health data may be conveniently displayed for the user or another party. In other embodiments, a wired connection may be utilized for communication of health data between the display device 930 and the health monitoring device 920.

The transmission of data from the health monitoring device 920 to the display device 930 may occur automatically without the user needing to prompt the transmission. Because the transmissions in this embodiment are automatic, some mechanism may be used to turn on the transceiver 1029 of the health monitoring device 920 or otherwise indicate that automatic transmissions should begin. For example, in one embodiment, an on/off switch is provided on the health monitoring device 920 that allows the user to begin automatic transmissions of data from the health monitoring device 920. In another example, the health monitoring device 920 may be configured to begin transmissions once it receives a confirmation that the display device 930 is within an appropriate range of the health monitoring device 920. In yet another embodiment, data transmission may occur periodically at predetermined intervals of time. In other embodiments, where communications between the health monitoring device 920 and the display device 930 are made with a wired connection, communications only occur when the wired connection is established between the health monitoring device 920 and the display device 930.

The health data transmitted to the display device 930 may be processed to determine one or more personal metrics for the user. As noted above, any of various personal metrics may be presented depending on the activity data or other health data collected by the health monitoring device 920. For example, the personal metrics may include, heart rates, awake times, sleep times, total steps, intensity level, sleep quality, calories spent, weight, body fat percentage, etc. The personal metrics may provide instantaneous activity information (e.g., current heart rate) or activity information determined over a given period of time (e.g., average heart rate). If the activity data indicates that the user is walking or running, the appropriate processor 1027 or 1037 may determine that the user is participating in a high intensity awake activity and/or may calculate a value for the intensity level. On the other hand, if the activity data indicates that the user is sitting or generally sedentary, the appropriate processor 1027 or 1037 may determine that the user is participating in a lower level awake activity. The activity data may automatically indicate that the user is sleeping or has retired to bed for an evening. In another example, the user may indicate on the health monitoring device 920 and/or on the display device 930 that he or she has retired to bed. During these times, the appropriate processor 1027 or 1037 may determine a quality of sleep of the user by determining activity levels during sleep. Relatively low movement and/or low heart rate during sleep may indicate deeper sleep levels and significant movement during sleep and/or increased heart rate may indicate lighter sleep or even additional awake times. When the user awakens the following morning, the appropriate processor 1027 or 1037 may automatically determine based on the activity signals that the user has awakened from his or her sleep and is participating in activities of various intensities.

After the activity data or other health data is processed to determine one or more personal metrics for the user, the processor 1037 may further process the health data in order to present the health data in a format for quickly and easily communicating the collected health data to the user. To this end, the processor is configured to communicate with the I/O interface 1036 and cause display of the processed activity or health information on the screen 934 for viewing by the user.

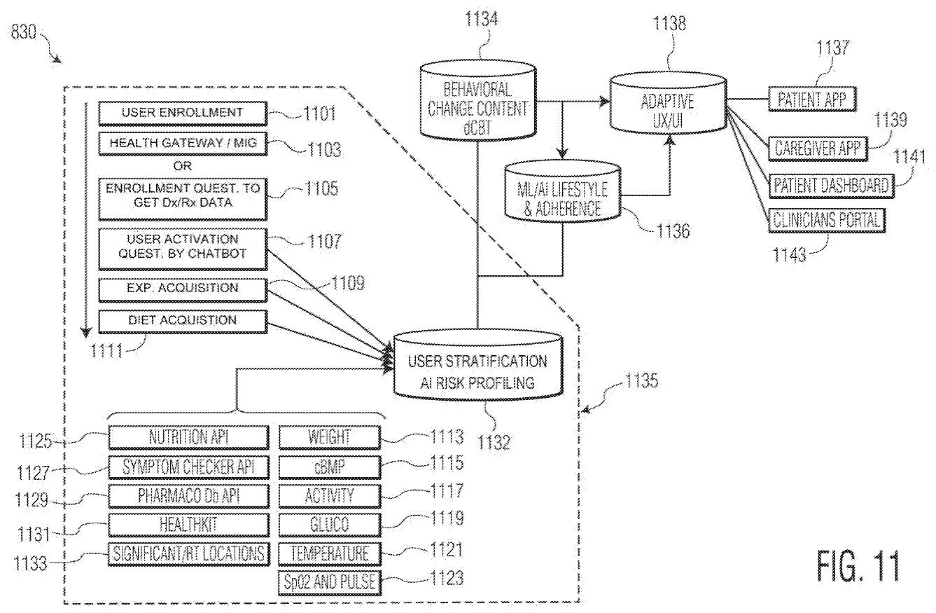

Referring now to FIG. 11, a functional diagram illustrating elements of the personalized medical system 830 is shown. The personalized medical system 830 may include one or more diagnostic and therapeutic components, such as a user stratification component 1132 to provide initial and incremental diagnosis of a user's status, a behavioral change content component 1134, a lifestyle and adherence component 1136, and adaptive conversational user interface and user experience (UI/UX) component 1138 to provide personalized condition treatment and management.

The user stratification component 1132, the behavioral change content component 1134, the lifestyle and adherence component 1136, and the adaptive conversational UI/UX component 1138 may be comprised of one or more software modules. The one or more software modules may be collections of code or instructions stored on a media (e.g., a memory of analytics server 822) that represent a series of machine instructions (e.g., program code) that implements one or more algorithmic steps. Such machine instructions may be the actual computer code a processor of the personalized medical system 830 interprets to implement the instructions or, alternatively, may be a higher level of coding of the instructions that is interpreted to obtain the actual computer code. The one or more software modules may also include one or more hardware components. One or more aspects of an example algorithm may be performed by the hardware components (e.g., circuitry) itself, rather as a result of an instruction.

The user stratification component 1132, the behavioral change content component 1134, the lifestyle and adherence component 1136, and the adaptive conversational UI/UX component 1138 may each include one or more of a natural language processor (NLP) device and a machine learning module. The NLP device may be configured to retrieve prepared data from the transaction database 824 shown in FIG. 8 and scan the prepared data to learn and understand the content contained therein. The NLP device may then selectively identify portions of the prepared data and extract certain features for preparation of a data set. The machine learning module may include one or more instructions to train a prediction model to generate information. To train the prediction model, the machine learning module may receive, as input, data from the one or more user devices 810 and or the repository 840 shown in FIG. 8. The machine learning module may implement one or more machine learning algorithms to train the prediction model to generate information. The machine learning module may use one or more of AB testing, a decision tree learning model, association rule learning model, artificial neural network model, deep learning model, inductive logic programming model, support vector machine model, clustering mode, Bayesian network model, reinforcement learning model, representational learning model, similarity and metric learning model, rule based machine learning model, and the like to train the prediction model.

The one or more diagnostic and therapeutic components of the personalized medical system 830 may communicate with the one or more user devices 810 during a course of treatment. The one or more diagnostic and therapeutic components may provide diagnostic tests to the one or more user devices 810 and may receive diagnostic feedback from the one or more user devices 810. The one or more diagnostic and therapeutic components may use the feedback to determine a diagnosis of a user. An initial diagnosis may be based on a comprehensive set of user data, tests, and questions, for example, while incremental updates may be made to a diagnosis using smaller data samples.

The one or more diagnostic and therapeutic components may communicate a diagnosis to the one or more user devices 810 of one or more of the patient, the caregiver, the health care provider, and the payor. The one or more diagnostic and therapeutic components may use the diagnosis to suggest therapies to be performed to treat any diagnosed symptoms. The one or more diagnostic and therapeutic components may send recommended therapies to the one or more user devices 810, including instructions for the patient and caregivers to perform the therapies recommended over a given time frame.

After performing the therapies over the given time frame, the caregivers or patient can indicate completion of the recommended therapies, and a report can be sent from the one or more user devices 810 to the one or more diagnostic and therapeutic components. The one or more diagnostic and therapeutic components may then indicate that the latest round of therapy is finished, and that a new diagnosis is needed. The one or more diagnostic and therapeutic components may then provide new diagnostic tests and questions to the one or more user devices 810, as well as take input from the therapy component of any data provided as part of therapy. The one or more diagnostic and therapeutic components may then provide an updated diagnosis to repeat the process and provide a next step of therapy.

The user stratification component 1132 may be part of the analytics server 822 shown in FIG. 8 and may work in conjunction with the analytics API gateway 820 and the web client application server 818. The user stratification component 1132 may utilize AI to coordinate user enrollment 1101 via one or more of a health gateway 1103 or an enrollment questionnaire 1105 that may be displayed on the one or more user devices 810. The enrollment questionnaire may be conducted by a conversational user interface through the application 802 displayed on the one or more user devices 810. The user stratification component 1132 may gather information about the user's experience and diet utilizing one or more APIs directed to, for example, diet/weight, symptoms, pharmacological history, health, and location. For example, the user stratification component 1132 may further utilize AI to conduct a user activation questionnaire 1107 and acquire information about a user's experience 1109 and diet 1111. As described above, the user stratification component 1132 may receive data from the one or more user devices 810, either entered directly by the user or by one or more of the sensors 1026 that record one or more of weight 1113, blood pressure 1115, activity 1117, blood glucose levels 1119, temperature 1121, and blood oxygen levels and pulse 1123.

The user stratification component 1132 may predict cognitive behavioral therapy acceptance and assess one or more of treatment and lifestyle faults (e.g., nutrition, exercise, weight). Using one or more of the repositories 840, the user stratification component 1132 may assess and validate adherence status to the treatment. For example, the user stratification component 1132 may be connected to one or more APIs to allow a user to access one or more of a nutrition tracker 1125, a symptom checker 1127, a pharmacological database 1129 to check for drug characteristics and drug-drug and/or drug-food interactions, Healthkit 1131, a location tracker 1133, and an adverse events (AE) checker to determine if AE is influencing adherence. The user stratification component 1132 may create an anonymous risk profile for the user and transmit it to the behavioral change content component 1134.

As shown in FIG. 11, all data gathered and processed by the user stratification component 1132 may be protected 1135 by Blockchain technology to ensure security and user-ownership of the data. In another example, data may remain encrypted on the one or more devices 810.

The behavioral change content component 1134 may be part of the analytics server 822 shown in FIG. 8 and may work in conjunction with the analytics API gateway 820 and the web client application server 818. The behavioral change component 1134 may interact with one or more repositories 840 and use a variety of behavior change techniques and associated tools to influence user behavior through the application 802. For example, the behavioral change component 1134 may generate instructions for a user to teach a particular behavior. The behavioral change component 1134 may encourage a user to self-monitor (e.g., record a behavior, write in a food diary) to assist in behavior change. The behavioral change component 1134 may assist in relapse prevention (e.g. by assisting with problem solving and identifying coping strategies). The behavioral change component 1134 may encourage engagement of social support (e.g., from family members). The strategy and content deployment utilized by the behavioral change component 1134 may be informed/triggered by the risk profile that is derived by the user stratification component 1132.

In an example, the behavioral change component 1134 may facilitate transactions between one or more of patients, providers, research organizations, etc. The transactions may utilize any type of currency, including a nonfungible token (NFT) and/or a cryptographic (crypto) coin. In one example, a specialized crypto coin and/or token may be used. The specialized crypto coin may provide one or more functions for users, such as: fees for access to medical data, rewards for providing medical and lifestyle data, rewards for achieving improved outcomes through behavior change, staking upon registration for self-sovereign identity, staking also for access to certain data sets, and acquiring NFT digital medical certificates. The specialized crypto coin and/or token may drive user engagement through the rewarding of behavior change (which may lead to better patient outcomes) and for providing access to medical and lifestyle data.

The specialized crypto coin and/or token may be secured via blockchain and may be managed by the user stratification component 1132. The specialized crypto coin may include consent management though one or more of patient, caregiver, clinician, and provider smart contracts. A patient and/or caregiver may send rights management transactions with identification though blockchain when he/she sets up or changes the settings for their information privacy. A clinical may active smart contracts that send information to the myAVOS platform and generate log entries and save to a blockchain database. Providers and researchers may active smart contracts that retrieve information from the myAVOS platform, generate log entries and save to the blockchain database, and activate a payment process.

A wallet of one or more of the patient, caregiver, and clinician may receive the payment in the specialized crypto coin via the blockchain (with a coin ID and a timestamp) for granting access to their information and/or when improved lifestyle and health outcomes are achieved.

When patients and/or caregivers upload and/or download medical records with identification, the data may be uploaded to a secure HIPAA and GDPR compliant system which contains demographic data as well as lifestyle and medical information. The data may then be encrypted and saved in the secure database. At the same time, a transaction record is saved to the blockchain with an identification, IP address, and date/time,

When a clinician uploads and/or downloads anonymized medical data, they may retrieve it from the secure server. The data may be decrypted (e.g., with a doctor's authorization) as recorded in the private blockchain. The clinician may upload data to the secure server, which is then encrypted and saved in the secure database. Authorization for the above upload and retrieval may be provided to the clinician under the rights management access authorized by the user. At the same time, a transaction record is saved to the blockchain including identification, IP address, and date/time.

Scientific researchers and pharmaceutical companies may retrieve de-identified data from the blockchain. When this occurs, a transaction record may be saved to the blockchain including an identification, IP address, and date/time.

The lifestyle and adherence component 1136 may be part of the analytics server 822 shown in FIG. 8 and may work in conjunction with the analytics API gateway 820 and the web client application server 818. The lifestyle and adherence component 1136 may use deep learning (DL) to improve the content generated by the behavioral change content component 1134 in conjunction with the one or more repositories 840. The lifestyle and adherence component 1136 may learn from input received from the user stratification component 1132 and the behavioral change content component 1134, aiming to provide better guidance than the human designed content generated by the behavioral change content component 1134. The lifestyle and adherence component 1136 may gather enough data to operate independently from the behavioral change content component 1134.

The adaptive UI/UX component 1138 may generate elements of the application for display on or more of the patient application 1137, caregiver application 1139, patient dashboard 1141, and clinician's portal 1143 on the one or more user devices 810 and receive input from the same. The adaptive UI/UX component 1138 may generate the conversational user interface/virtual coach to provide users with lifestyle guidance and a medication coach. The adaptive UI/UX component 1138 may assist in the preliminary diagnosis/triage of users and may generate one or more of a dashboard, notifications, prescription scheduling, live chat, and live video conferencing. In an example, the adaptive UI/UX component 1138 may use deep fake technology so that the conversational user interface may look like a real human being.

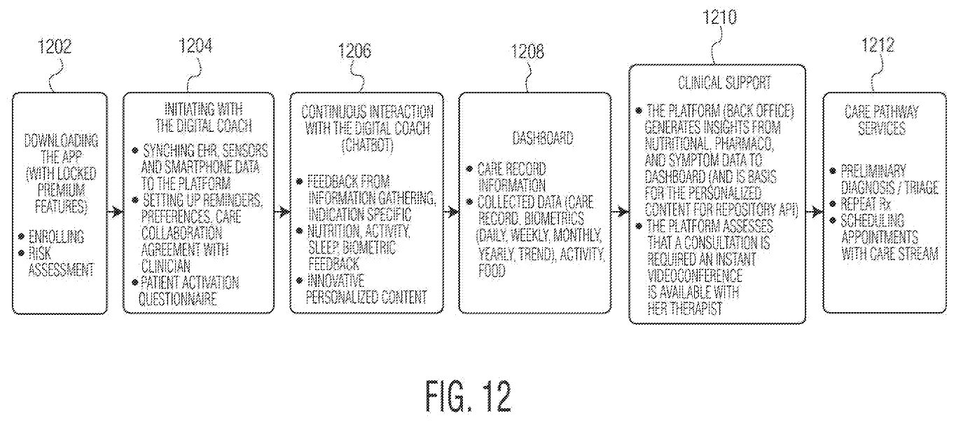

Referring now to FIG. 12, a flowchart showing an overall flow of patient treatment is shown. In Step 1202, a user may download the application 802, which may include one or more premium features to be unlocked by the user. The user may enroll in the application 802 and may complete a risk assessment. In Step 1204, the user may initiate the digital coach. The platform may synchronize one or more of the user's EHR, and data received from the sensors 1026 and/or input into the one or more user devices 810. The platform may set up reminders, user preferences, and a care collaboration agreement with a clinician. The user may complete an activation questionnaire through the application 802. In Step 1206, the platform may provide continuous interaction with the user through the conversational user interface. The platform may provide feedback from information gathered form the user and may provide innovative personalized content. In step 1208, the platform may provide a dashboard to the user with care record information and additional information on collected data. In Step 1210, the platform may generate insights from the user's nutritional, pharmacological, and symptom data and present these insights on the user's dashboard. If the platform determines that a consultation is required, it may initiate a videoconference between the patient and a caregiver. In Step 1212, the platform may provide one or more care pathways to the patient.

The platform may include a single mobile application 802 that may be used by, at least, patients and caregivers to treat and manage chronic conditions. The caregiver may be, for example, assigned the caregiver role by the patient through the application 802. The caregiver may have access to patient data and may receive in-application notification alerts specified by the application 802.

The patient-facing application 802 may allow patients to continuously assess their lifestyle and medical regimes. The patient-facing application 802 may be available on multiple platforms and may support multiple languages. To enroll, patients may be able to create a new account using an email and password or may log in using an existing third-party account. The application 802 may issue an initial test/questionnaire to assess the patient and may interface with one or more third party systems to obtain the patient's EHR, including one or more of: current diagnosis, current problems, current medication, past diagnosis, medication issues, operations, blood pressures, encounters, admissions, referrals, and the like.

The patient-facing application 802 may integrate one or more digital health services (e.g., HealthKit and GoogleFit) and health monitoring devices 920 to gather data about one or more of the following: body measurements, height, weight, body fat percentage, body mass index, lean body mass, waist circumference, heart data, blood pressure, ECG measurements, heart rate, heart rate variability, high heart rate notifications, irregular heart rate notifications, low hear rate notifications, rest heart rate, walking heart average, reproductive health, basal body temperature, cervical mucus quality, menstruation, ovulation test results, sexual activity, spotting, blood alcohol content, blood glucose, electrodermal activity, forced expiratory volume, forced vital capacity, inhaler usage, insulin delivery, number of times fallen, oxygen saturation, peak expiratory flow rate, peripheral perfusion index, UX index, mindfulness, mindful minutes, activity, steps, activity/active energy, cycling distance, downhill snow sports distance, exercise minutes, flight minutes, Nike Fuel, pushes, resting energy, standing hours, swimming distance, swimming strokes, VO2 max, wheelchair distance, medical ID, photographs, date of birth, medical conditions, medical notes, allergies & reactions, medications, blood type, donor organs, emergency contacts, nutrition, biotin, caffeine, calcium, carbohydrates, chloride, chromium, copper, dietary cholesterol, dietary energy, dietary sugar, fiber, folate, iodine, iron, magnesium, manganese, molybdenum, monounsaturated fat, niacin, pantothenic acid, phosphorus, polyunsaturated fat, potassium, protein, riboflavin, saturated fat, selenium, sodium, thiamin, total fat, vitamin A, vitamin B12, vitamin B6, vitamin C, vitamin D, vitamin E, water, zinc, and sleep.

The patient-facing application 802 may provide information on the patient's medications and any potential interactions. By interfacing with one or more third-party services, the patient-facing application 802 may allow a patient to perform one or more of the following: search medicine by name, search medicine by active component, see medications, doses, side effects, see the list of drugs from current medications, see detailed information on the selected drug from current medication, see dosage form, see delivery route, see drug name, see simple description, see food interactions, and see strength number and unit. A patient may see recommendations and warnings about the interaction between medications, see the severity of the interaction effect between the drugs, obtain the interaction information for two or more drugs, and obtain management information to prevent the drug interaction effect.

The patient-facing application 802 may provide nutrition information. By interfacing with one or more third-party services, the patient-facing application 802 may allow a patient to perform one or more of the following tasks: find nutrition by name of product, find nutrition by scanning a barcode, calculate needed calories per day, track calories per day measurements over time, and track individual nutrients.

The patient-facing application 802 may provide symptom checking, using for example, the conversational user interface feature. The conversational user interface may ensure that relevant biometric data and digital biomarker data are input and may be used to set goals (weight, activity, calories, etc.). The conversational user interface may provide visually attractive data feedback around wellness data (activity, sleep, diet, etc.) and may provide content relevant to the current status of the patient so that behavioral modification is effective. The conversational user interface may initiate a suggestion on the basis of current status and may schedule an appointment with the patients' care team.

The patient-facing application 802 may provide geo-fencing and tracking. The patient may be able to set a geofence and add caregiver contacts for notifications. The patient-facing application 802 may track patient location and alert a caregiver if the patient leaves the specified area.

The patient-facing application 802 may provide a patient dashboard that displays one or more of goal metrics, a steps chart, a distance chart, calories burned chart, and a calendar. The patient-facing application 802 may allow a patient to book appointments with caregivers and may provide cognitive stimulation. The patient-facing application 802 may provide one or more notifications to the patient, such as: weight input reminder, nutrition input reminder, pills and medicine intake reminders, cognition development reminders, activity reminders, blood pressure measurement reminders, and hydration reminders.

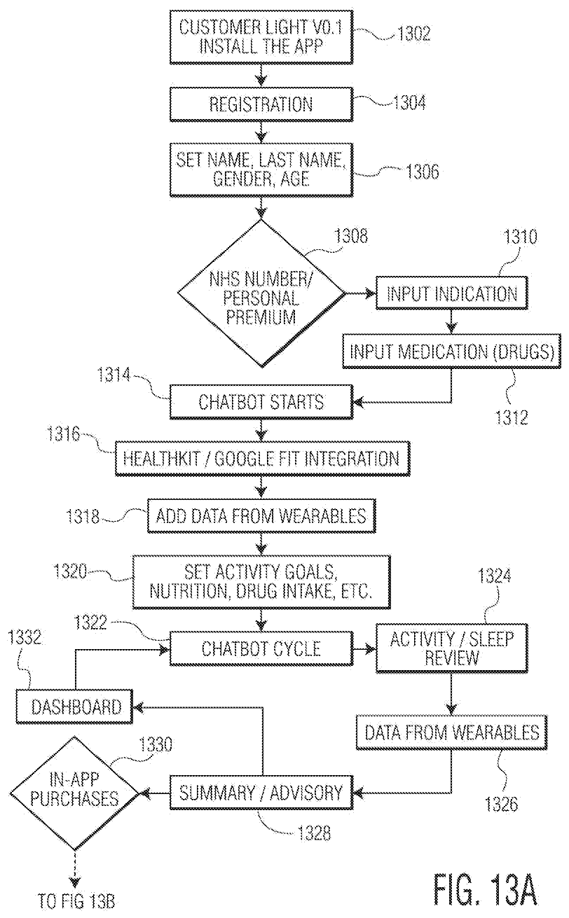

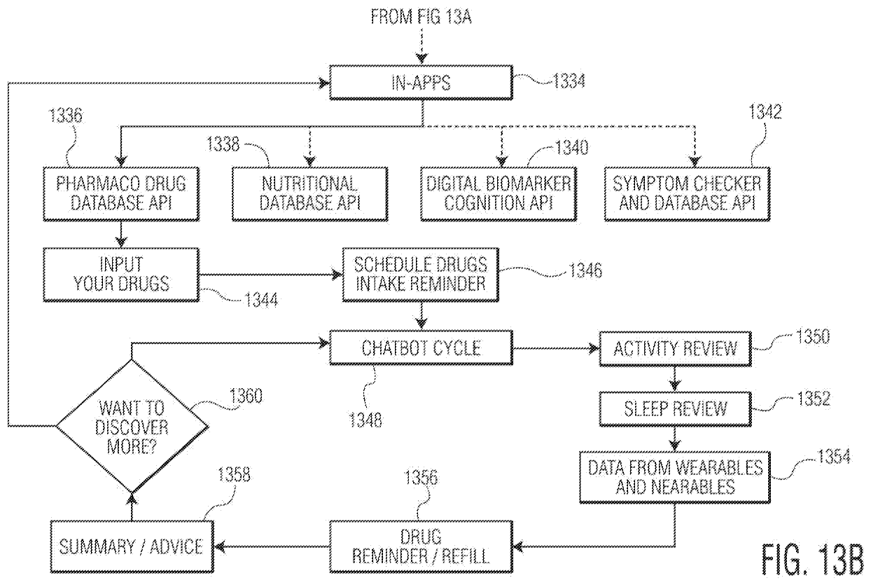

Referring to FIGS. 13A-13C a flowchart illustrating the registration and use of the patient-facing application 802 provided by the platform is shown. FIGS. 13A-13C show a light version of the patient-facing application 802 that includes in-application purchases. In Step 1302, the patient may download the patient-facing application 802. In Step 1304, the patient may register with the patient-facing application 802. In Step 1306, the patient may enter his or her information. In Step 1308, the patient may enter their medical number. In Step 1310, patient may enter any indications. In Step 1312, the patient may enter medication information. In Step 1314, the conversational user interface may initiate interactions with the patient. In Step 1316, the platform may integrate with one or more health trackers. In Step 1318, the platform may incorporate data from one or more wearables and sensors. In Step 1320, the platform may set patient parameters.

In Step 1322, the conversational user interface cycle may initiation. In Step 1324, the platform may track activity/sleep. In Step 1326, the platform may receive additional data from the one or more wearables and sensors. In Step 1328, the platform may generate a summary/advisory. If the patient does not have an additional add-in, in Step 1332 the summary/advisory may be provided to the dashboard. If the patient has made in-application purchases, the process proceeds to Step 1330.

In Step 1334, the platform may interact with one or more repositories 840 as described above. For example, the platform may interact with one or more of a pharmaco-drug database API 1336, a nutritional database API 1338, a digital biomarker cognition API 1340, and a symptom checker and diagnosis API 1342. In Step 1344, the platform may receive drug information for the patient. In Step 1346, the platform may generate a schedule and reminders to take medications. In Step 1348, the conversational user interface cycle may be re-initiated. In Step 1350, the platform may review the patient's activity. In Step 1352, the platform may review the patient's sleep. In Step 1354, the platform may gather additional information from the one or more wearables and sensors. In Step 1356, the platform may generate a drug/refill reminder. In Step 1358, the platform may generate a summary/advice for the patient. At Step 1360, the platform may generate a prompt for the user to add additional in-application purchases.

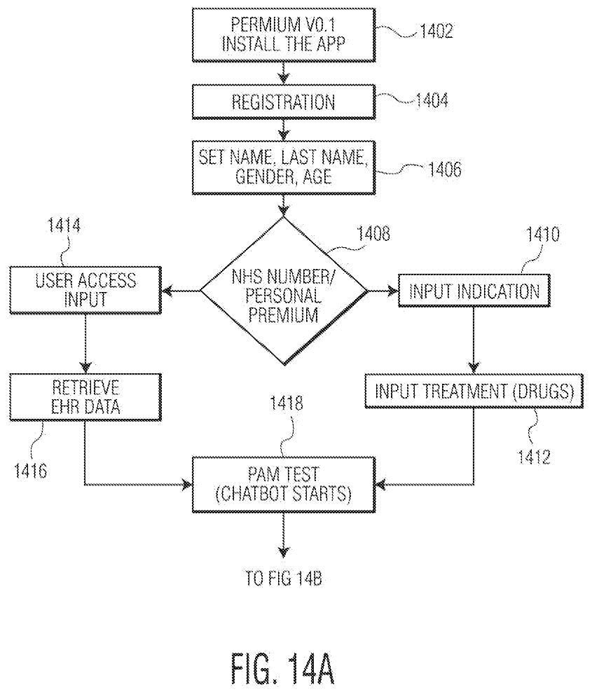

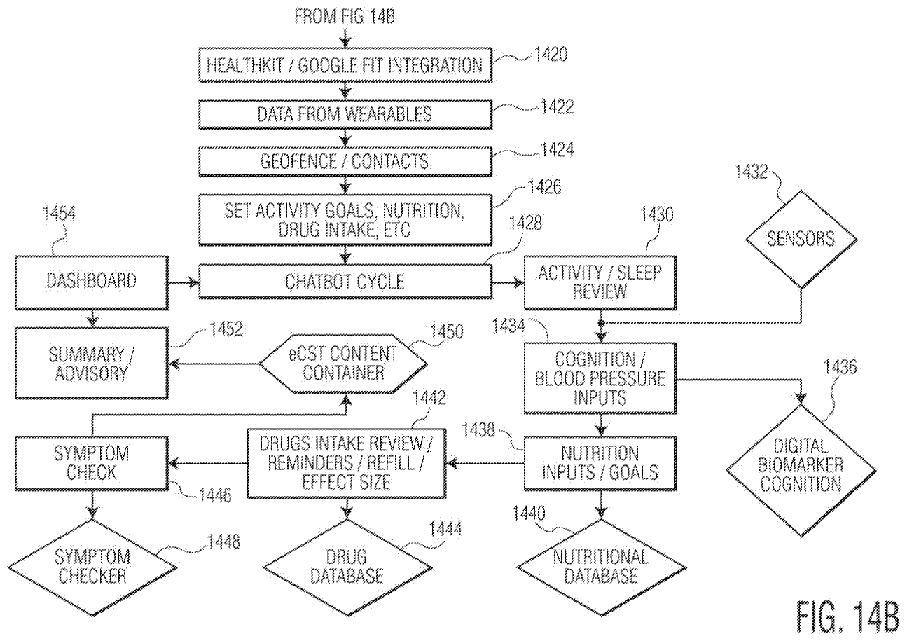

Referring to FIGS. 14A-14B a flowchart illustrating the registration and use of the patient-facing application 802 provided by the platform is shown. FIGS. 14A-14B show a premium version of the application 802 with all features unlocked. In Step 1402, the patient may download the patient-facing application 802. In Step 1404, the patient may register with the patient-facing application 802. In Step 1406, the patient may enter his or her information. In Step 1408, the patient may enter their medical number. In Step 1410, patient may enter any indications. In Step 1412, the patient may enter medication information. In Step 1414, the patient may grant the platform access to their EHR records. In Step 1416, the platform may retrieve the patient's EHR records. In Step 1418, the conversational user interface may initiate interactions with the patient.

In Step 1420, the platform may integrate with one or more health trackers. In Step 1422, the platform may incorporate data from one or more wearables and sensors. In Step 1424, the patient may establish a geofence and enter contacts to be notified. In Step 1426, the platform may set activity goals, nutrition, and drug intake. In Step 1428, the conversational user interface cycle may reinitiate. In Step 1430, the platform may review the patients sleep/activity. In Step 1432, sensor data may be incorporated. In Step 1434, cognition and/or blood pressure inputs may be received. In Step 1436, the platform may interface with a repository 840, such as a digital biomarker cognition API. In Step 1438, the platform may receive nutrition inputs and goals. In Step 1440, the platform may interface with a repository 840, such as a nutritional database API. In Step 1442, the platform may review drug intake and may provide information/reminders. In Step 1444, the platform may interface with a repository 840, such as pharmaco-drug database API. In Step 1446, the platform may initialize a symptom check. In Step 1448, the platform may interface with a repository 840, such as a symptom checker API. In Step 1450, the platform may initiate an eCBT content container. In Step 1452, the platform may generate a summary/advisory. In Step 1454, the summary/advisory may be provided to the dashboard and the process may return to Step 1428, where the conversational user interface cycle may reinitiate.

The caregiver-facing application 802 may allow caregivers to continuously assess the patient's lifestyle and medical regimes. A patient may have the ability to add a caregiver's name, last name, phone number, email into the patient-facing application 802 and send an invitation link to the caregiver. The caregiver may receive a link to install the caregiver-facing application 802 and/or add the patient to their previously installed caregiver-facing application 802.

The caregiver-facing application 802 may allow a caregiver to create an account via email and password or through a third-party account. A caregiver may have access to one or more patients and may select an individual patient to see their profile. The caregiver may be able to obtain the patient's data, including one or more of the current state of the patient (e.g., performance, recommendations, dashboard), their activity goals and statistics, their medicine list, their medicine intake, their biometric data, nutrition, and geofence. The caregiver-facing application 802 may generate alerts if one or more of the following occurs: the patient has left the geofence area, the patient hasn't moved for a predetermined period of time, the patient has missed a medicine intake, and the user did not complete a personal activity goal. The caregiver-facing application 802 may allow the caregiver to actively monitor one or more patients and may help manage caregiver wellbeing and stress.

In summary, the novel myAVOS platform described above may significantly optimize the patient/caregiver journey through disease treatment/management. During pre-diagnosis and diagnosis stages, myAVOS may provide earlier and faster identification of diseases through the use of (digital) biomarkers, screening, lifestyle interventions, and enrollment in clinical trials. During the initiation of treatment and the continuing treatment stages, myAVOS may optimize treatment based on real time data from wearables and other sensors resulting in the continued monitoring of treatment effectiveness and improvement in resource utilizations. The myAVOS platform may also increase adherence and engagement through behavioral change content, which may result in optimized treatment adherence and effectiveness, reduction in burdens on caregivers, and a delay in admission to supervised care facilities. The myAVOS platform may reduce disease severity and/or rate of decline in patients, reduce burdens on caregivers, prevent and mitigate critical wandering, and improve adherence to treatment/management plans.

Although features and elements are described above in particular combinations, one of ordinary skill in the art will appreciate that each feature or element can be used alone or in any combination with the other features and elements.

What is claimed is:

1. A system for the remote screening, diagnosis, monitoring, and remote management of one or more chronic health conditions, the system comprising:
a processor; and
a memory having programming instructions stored thereon, which, when executed by the processor, performs one or more operations comprising:
registering a patient and a caregiver with the system via a single application;
retrieving an electronic health record (EHR) for the patient from one or more repositories;
receiving patient information from the patient via a conversational user interface displayed on the patient's application;
receiving health data from one or more patient devices;
generating a predictive model for actionable insights for one or more of the patient and the caregiver using one or more machine learning models by:
generating a risk profile for the patient based on one or more of the EHR, the patient information, the data, and information from the one or more repositories, and
generating one or more behavior change techniques based on the risk profile; and
delivering the one or more behavior change techniques to one or more of the patient and the caregiver as natural language messages via the conversational user interface.

2. The system of claim 1, wherein the generating the predictive model for actionable insights further comprises:
initiating a communication between the patient and the caregiver through the application.

3. The system of claim 2, wherein the communication comprises one or more of voice call, a chat message, and a videoconference.

4. The system of claim 1, wherein the patient information is input by one or more of the patient and the caregiver in response to an initial questionnaire.

5. The system of claim 1, wherein the patient information comprises information about one or more of the patient's diet, weight, activity levels, daily habits, symptoms, pharmacological history, health, and location.

6. The system of claim 1, wherein the health data comprises one or more of weight, blood pressure, activity, blood glucose levels, temperature, blood oxygen levels, sleep, and pulse.

7. The system of claim 1, wherein the one or more patient devices comprise a health monitoring device.

8. The system of claim 1, wherein the one or more repositories comprise a computer system storing one or more of EHR, nutritional databases, pharmacological databases, symptom diagnosis databases, biomarker databases, and therapeutic databases.

9. The system of claim 1, wherein the generating the predictive model for actionable insights further comprises:
continuously monitoring one or more of the patient's cognition, treatment effect, and health data based on the risk profile;
receiving input from the patient via one or more of the application and the one or more patient devices; and
generating one or more recommended remote interventions for one or more of the patient and the caregiver.

10. The system of claim 1, wherein the application comprises a dashboard for one or more of the patient and the caregiver that displays one or more of care record information, collected information, and recommendations.

11. A method for screening, diagnosis, and management of one or more chronic health conditions, the method comprising:
registering a patient and a caregiver with an electronic health system via a single application;
retrieving an electronic health record (EHR) for the patient from one or more repositories;
receiving patient information from the patient via a conversational user interface displayed on the patient's application;
receiving health data from one or more patient devices;
generating a predictive model for actionable insights for one or more of the patient and the caregiver using one or more machine learning models by:
generating a risk profile for the patient based on one or more of the EHR, the patient information, the data, and information from the one or more repositories, and
generating one or more behavior change techniques based on the risk profile; and
delivering the one or more behavior change techniques to one or more of the patient and the caregiver as natural language messages via the conversational user interface.

12. The method of claim 11, wherein the generating the predictive model for actionable insights further comprises:
initiating a communication between the patient and the caregiver through the application.

13. The method of claim 12, wherein the communication comprises one or more of a voice call, a chat message, and a videoconference.

14. The method of claim 11, wherein the patient information is input by one or more of the patient and the caregiver in response to an initial questionnaire.

15. The method of claim 11, wherein the patient information comprises information about one or more of the patient's diet, weight, activity levels, daily habits, symptoms, pharmacological history, health, and location.

16. The method of claim 11, wherein the health data comprises one or more of weight, blood pressure, activity, blood glucose levels, temperature, blood oxygen levels, sleep, and pulse.

17. The method of claim 11, wherein the one or more patient devices comprise a health monitoring device.

18. The method of claim 11, wherein the one or more repositories comprise a computer system storing one or more of EHR, nutritional databases, pharmacological databases, symptom diagnosis databases, biomarker databases, and therapeutic databases.

19. The method of claim 11, wherein the generating the predictive model for actionable insights further comprises:
continuously monitoring one or more of the patient's cognition, treatment effect, and health data based on the risk profile;
receiving input from the patient via one or more of the application and the one or more patient devices; and
generating one or more recommended remote interventions for the patient.

20. The method of claim 1, wherein the application comprises a dashboard for one or more of the patient and the caregiver that displays one or more of care record information, collected information, and recommendations.
